(19) United States
(12) Patent Application Publication
(10) Pub. No.: US 2015/0275231 A1
(43) Pub. Date: Oct. 1, 2015

(54) Method of Preventing or Reducing Virus Transmission in Animals

(71) Applicant: Mice With Horns, LLC, Delray Beach, FL (US)

(72) Inventor: James WEST, Nashville, TN (US)

(21) Appl. No.: 14/675,135

(22) Filed: Mar. 31, 2015

Related U.S. Application Data
(60) Provisional application No. 61/972,745, filed on Mar. 31, 2014.

(51) Int. Cl. C12N 15/85; A01K 67/027; C12N 15/90; A61K 35/741

(52) U.S. Cl. CPC C12N 15/8509; A61K 35/741; A01K 67/0278; C12N 15/907; A01K 2207/15; A01K 2217/072; A01K 2227/108; A61K 2035/115

(57) ABSTRACT

The subject invention provides materials and methods for improving animal resistance to infection by intestinal viruses. This is accomplished by interfering with intestinal virus uptake employing methods that (1) reduce virus binding to receptors in the intestinal lining; (2) introduce decoy receptors expressed in the mammary gland leading to decoy secretion in milk; (3) produce decoy receptors by a variety of protein synthesis methods to provide decoy receptors to non-genetically modified animals, including humans; and/or (4) administer a vector to a non-genetically modified animal which vector has been genetically modified to produce a decoy receptor.

### CROSS-REFERENCE TO RELATED APPLICATION

This application claims the priority benefit of U.S. Provisional Application Ser. No. 61/972,745, filed Mar. 31, 2014, which is incorporated herein by reference in its entirety.

The Sequence Listing for this application is labeled SEQ-LIST-3-27-15-ST25.txt which was created on Mar. 27, 2015 and is 9 KB. The entire content of the sequence listing is incorporated herein by reference in its entirety.

### BACKGROUND

Viruses, including coronaviruses and noroviruses, are a significant source of morbidity and mortality in both the livestock industry and in humans. For example, Porcine Epidemic Diarrhea (PED) virus kills millions of piglets per year, with a mortality of 50% in infected litters, and an annual industry cost in the billions of dollars.

While some viruses work across species, and have very promiscuous targets, the majority of intestinal viruses, including PED, have high specificity. They recognize a specific receptor, and only the version of that receptor found in one species. The variation in the specific receptor across species is a large part of the origin of species specificity.

Intestinal viruses are difficult to block, for several reasons. Because of their mutation rate, vaccines are only useful for one season—and because the viruses infect through the intestinal lining, vaccines can reduce the intensity of the disease, but cannot reduce initial infection, because the more effective IGG and IGM antibodies produced by the vaccine are only in the bloodstream, not the intestinal space. There are no therapeutics agents currently targeted at intestinal viruses.

One method of blocking intestinal viruses is found in nature—human breast milk appears to contain decoy receptors to some classes of norovirus. Nursing babies thus have substantial protection, because any virus they ingest targets the decoy receptors rather than the receptors in the infant's intestines.

### BRIEF SUMMARY

The subject invention provides materials and method for improving animal resistance to infection by viruses. In preferred embodiments, this is accomplished by interfering with virus uptake employing methods that (1) reduce virus binding to receptors on cells that are naturally infected by such virus, (2) introduce a decoy receptor gene into the genome of an animal to induce secretion of a decoy receptor, (3) administer one or more decoy receptors to an animal, and/or (4) administer a vector to an animal wherein the vector has been genetically modified to produce a decoy receptor.

### BRIEF DESCRIPTION OF THE SEQUENCE

SEQ ID NO:1 shows the sequence of ANPEP of Bactrian camels for use according to the method of the subject invention.

### DETAILED DISCLOSURE

The subject invention provides materials and method for improving animal resistance to infection by viruses. The resistance to viral infection is achieved according to the subject invention by preventing virus binding to specific receptors naturally found in the animal to be protected from infection.

Prevention of virus binding is achieved by either customizing animals to express receptors that do not lead to infection by such viruses, and/or by providing decoy receptors that efficiently bind viruses within body fluids and/or a body cavity and block viral uptake through the lining of such fluid compartment or cavity.

In one embodiment, a decoy receptor derived from a receptor that naturally functions in molecule transport across a cell membrane and is used by viruses to facilitate virus infection, is modified such that receptor binding to its natural target molecule is reduced and the binding to the viruses remains unchanged.

In cases where the virus-binding amino acids on a cellular receptor are known, a decoy of a receptor can be generated that lacks most or all of the amino acids involved in interaction with the virus.

Decoy receptors can also be produced through several methods of protein synthesis, including production in bacteria, yeast, viruses or production through artificial synthesis methods. The decoy receptors and/or the virus, bacterium, or other microbe that expresses the decoy receptors are then stored and can be administered, for example, during a viral outbreak to reduce susceptibility of decoy-receiving animals to viral infection. This method allows protection for non-genetically modified animals, including humans.

In embodiments specifically exemplified herein, the natural infection of animals by viruses is abrogated by replacing a cell membrane receptor that is susceptible to virus infection with a cell membrane receptor that, when bound by a virus, does not lead to a pathological infection in the animal. The subject invention provides materials and methods for customizing animal cellular receptor expression, wherein the methods of the invention can utilize knowledge of cellular receptors for pathogenic viruses, targeted gene modification, and preferably, spermatogonial stem cell (SCC) transfer to facilitate production of virus resistance-customized sperm.

In preferred embodiments, the virus infection-susceptible endogenous receptor in animals can be replaced with a receptor from another species with either no known susceptibility to infection by such viruses, or with which animals of the subject invention are unlikely to come in contact.

In one embodiment, the endogenous nucleic acid encoding the virus infection-susceptible endogenous receptor is in the genome of the animal. In one embodiment, the genome of at least one SSC comprises a replaced nucleic acid molecule that does not contain the undesirable virus infection-susceptible endogenous receptor.

In embodiments specifically exemplified herein, the natural infection of animal intestines by intestinal viruses is abrogated by replacing intestinal receptors that are susceptible to intestinal virus infection with intestinal receptors, which, when bound by a virus do not lead to a pathological infection in the animal.

In one embodiment, the subject invention provides a method for replacing the ANPEP gene, which encodes a receptor that binds Porcine Epidemic Diarrhea (PED) virus in pigs, with ANPEP from an alternative species such as Bactrian camels, woolly mammoths, tree sloths, giant pandas, or any other species that, because of the rarity or the environment in which the species lives, does not have known intestinal viruses.

In a preferred embodiment, both copies of the porcine ANPEP gene are replaced with ANPEP of the alternative species and the transgenic pigs become immune to PED virus infection, because the PED virus only recognizes the porcine version of the ANPEP receptor, which will be missing in these animals.

The invention further provides materials and methods to protect an animal from infection by a virus that infects through a receptor on the surface of a tissue, for example, a mucus membrane. Non-limiting examples of routes of viral entries or infection in an animal include the respiratory tract, conjunctiva, alimentary tract, urogenital tract and skin. Typically, these routes of viral infection involve a mucus membrane.

Accordingly, in one embodiment, the invention provides materials and methods to produce an animal resistant to infection by a virus that infects though a mucus membrane, wherein the mucus membrane:

a) expresses a mutant viral receptor to which the virus cannot bind,

b) expresses a homolog of a viral receptor from a species of animal that the virus cannot infect,

c) expresses a decoy receptor,

d) expresses a decoy receptor specifically in the cells of the mucus membrane, or

e) has associated therewith a vector that produces a decoy receptor.

The vector may be a virus, bacterium or other microbe. In preferred embodiments, the vector is a bacterium.

In preferred embodiments of the subject invention, a mutant viral receptor is a receptor to which a virus cannot bind; however, the mutant viral receptor provides the biological function of the non-mutated or wild type viral receptor. An example of a mutant viral receptor that could be used according to the invention is a mutant viral receptor comprising mutations in the amino acid residues that constitute the binding site for the virus so that the virus cannot bind to the receptor. Another example of a mutant viral receptor to which a virus cannot bind is a receptor lacking the portion of the receptor involved in viral binding. Additional examples of mutations that modify a receptor in a manner that the virus cannot bind to the receptor are well known to a person of ordinary skill in the art and such embodiments are within the purview of the invention.

In one embodiment, the animal resistant to the infection by a virus expressing a mutant viral receptor to which the virus cannot bind or a homolog of a viral receptor from a species of animal that the virus cannot infect is a transgenic animal containing one or more copies of the genes encoding the mutant viral receptor or the homolog of a viral receptor incorporated into the genome of the animal. In one embodiment, fragments of the homolog from an animal naturally resistant to the viral infection are used.

In another embodiment, one or both copies of the genes encoding the viral receptor in the genome of the animal are replaced by one or two genes encoding the mutant viral receptor or the gene encoding the homolog of a viral receptor.

In a further embodiment, the animal resistant to the infection by a virus expressing a decoy receptor is a transgenic animal containing one or more copies of the genes encoding the decoy receptor in the genome of the animal.

In a particular embodiment, the animal resistant to infection by a virus expresses a decoy receptor specifically in the cells of a mucus membrane through which the virus infects via one or more copies of the genes encoding the decoy receptor incorporated into the genome of the animal wherein the one or more genes encoding the decoy receptor are under the control of a promoter specific for the cells of the mucus membrane through which the virus infects.

In certain embodiments, the genetically engineered animal resistant to viral infection is a mammal. The mammal can be, for example, an ape, pig, canine, feline, or cattle.

A person of ordinary skill in the art can apply the methods and materials of the invention to produce any animal resistant to any virus based on the principles and embodiments described herein and the knowledge common in the art.

For example, to produce an animal resistant to a viral infection, the route of infection of the virus and the receptor involved in the infection of the susceptible animal is determined. The amino acids involved in the binding of the virus on the receptor are identified and a mutant viral receptor containing mutations in the amino acids that constitute the binding site is created. Alternately, an animal that is naturally resistant to the infection by the virus is identified and a homolog of the viral receptor in the naturally resistant animal is identified.

The infection-susceptible animal can then be genetically engineered to replace one or both copies of the viral receptor in the genome of the animal with one or two copies of the mutant viral receptor or with one or two copies of the viral receptor homolog.

In a particular embodiment, an animal resistant to a viral infection through the respiratory tract is created. An example of an animal susceptible to a virus that infects through the respiratory tract is a pig and non-limiting examples of viruses that infect pigs through the respiratory tract are swine influenza virus (SIV), porcine reproductive and respiratory syndrome virus (PRRSV), pseudorabies virus (PRV), porcine respiratory coronavirus (PRCV), porcine cytomegalovirus (PCMV), porcine paramyxovirus (PPMV), hemagglutin atingencephalomyelitis virus (HEV), encephalomyocarditis virus (EMC), porcine parvovirus (PPV), porcine adenovirus. Viral receptor homologs can be obtained from animals resistant to infection by these viruses, for example, Bactrian camels, woolly mammoths, tree sloths, giant pandas, or any other species that are resistant to a virus against which a resistant pig is to be produced.

A further embodiment of the invention provides an animal resistant to infection by a virus that infects through a mucus membrane, wherein the animal comprises, within the mucus membrane, a vector that produces a decoy receptor. The vector can be, for example, a non-pathogenic virus or bacterium, for example, a bacterium belonging to a normal microflora of the animal and the mucus membrane.

The invention also provides a method of producing an animal resistant to infection by a virus that infects though a mucus membrane. The method comprises modifying the animal so that the mucus membrane of the animal:

a) expresses a mutant viral receptor to which the virus cannot bind,

b) expresses a homolog of a viral receptor from a species of animal which the virus cannot infect,

c) expresses a decoy receptor,

d) expresses a decoy receptor specifically in the cells of the mucus membrane, or

e) has associated therewith a vector that produces a decoy receptor.

In one embodiment, the resistant animal is a genetically engineered animal.

In a certain embodiment, the genetically engineered animal is produced by introducing one or more copies of the genes encoding the mutant viral receptor or the homolog of a viral receptor incorporated into the genome of the animal.

In another embodiment, the genetically engineered animal is produced by replacing one or both copies of the genes encoding the viral receptor in the genome of the animal by one or two genes encoding the mutant viral receptor or the gene encoding the homolog of the viral receptor.

In a further embodiment, the genetically engineered animal is produced by introducing one or more copies of the genes encoding the decoy receptor into the genome of the animal.

In a certain embodiment, the genetically engineered animal is produced by introducing into the genome of the animal one or more genes encoding the decoy receptor that are under the control of a promoter specific for the cells of the mucus membrane.

The methods of genetically engineering animals, for example, creating a transgenic animal having tissue specific expression of the transgene, replacing one or both copies of a wild type gene in an animal with one or two copies of a mutant gene or a homologous gene are well known to a person of ordinary skill in the art.

In a further embodiment, transgenic pigs carrying the replaced ANPEP can be outbred and first-generation off-spring have one copy of the ANPEP gene originating from the alternative species, making such off-spring partially protected from PED virus infection.

In a preferred embodiment, the receptor sequence that interacts with the intestinal virus is specifically known, and only the nucleotides encoding the virus-interacting amino acids of the endogenous receptor are replaced with nucleotides from corresponding sequences of receptors of the alternative species.

In one embodiment, the natural infection of animal lungs by viruses is abrogated by replacing pulmonary receptors that are susceptible to virus infection with pulmonary receptors, which, when bound by a virus, do not lead to a pathological infection in the animal.

In a preferred embodiment, the receptors on alveolar macrophages targeted by PRRS virus (CD163 or CD169) are replaced with receptors from species that are not susceptible to infection by PRRS.

In preferred embodiments, the receptor genes from the alternative species express receptors that are fully functional in the intestine of the animal expressing the alternative species sequence despite the alternative receptors' inability to promote intestinal virus infection.

In a further embodiment, if more than one cellular receptor is used by a virus to infect an animal, all such cellular receptors are replaced by cellular receptors from alternative species that are not susceptible to infection by such virus.

In one embodiment, the cellular receptors known to be involved in pseudorabies infection are replaced with cellular receptors from species that are not susceptible to pseudorabies.

In one embodiment, the subject invention provides a method for replacing an undesirable nucleic acid sequence encoding a virus infection-susceptible endogenous cellular receptor with a desirable nucleic acid sequence encoding a virus infection-resistant receptor in animals, wherein the method comprises:

obtaining one or more spermatogonial stem cells (SSC) of a male animal that has a nucleic acid sequence encoding an undesirable virus infection-susceptible cellular receptor;

providing a replacement construct comprising an exogenous nucleic acid molecule for replacement of the undesirable nucleic acid sequence encoding a virus infection-susceptible cellular receptor with a desirable nucleic acid sequence encoding a virus infection-resistant receptor; and

introducing the replacement construct into at least one of the SSCs using a nuclease (such as a site-specific nuclease), thereby obtaining at least one corrected SSC comprising a replaced nucleic acid molecule that has the undesirable nucleic acid sequence replaced with the nucleic acid sequence encoding a virus infection-resistant cellular receptor; and optionally,

introducing one or more SSCs into a reproductive organ of a male recipient animal; and, optionally,

collecting the donor-derived, fertilization-competent, haploid male gametes produced by the male recipient.

In a preferred embodiment, both copies of the virus infection-susceptible endogenous receptor are replaced with the exogenous virus infection-resistant receptor.

In a preferred embodiment, the exogenous nucleic acid molecule encoding the virus infection-resistant exogenous receptor also contains inhibitory RNA nucleic acid sequences (miRNA) that target the RNA for the endogenous virus infection-susceptible receptor protein. In a preferred embodiment, the replacement construct containing the exogenous nucleic acid sequence encoding the virus infection-resistant receptor and one or more inhibitory RNA sequences targeting the RNA of endogenous virus infection-susceptible receptors in heterozygotes leads to suppression of virus infection-susceptible receptors and expression of virus infection-resistant exogenous receptors in outbred off-spring of homozygous males.

In a preferred embodiment, multiple miRNAs to the same gene are incorporated into the nucleic acid sequence of the subject invention thereby significantly enhancing knockdown of the endogenous virus-susceptible receptor gene. Thus, in one embodiment of the subject invention, multiple miRNAs that target a single receptor gene are provided in polycistronic strings.

In one embodiment, the subject invention provides a method for replacing the nucleic acid sequence encoding a naturally virus infection-susceptible cellular receptor in an animal with a nucleic acid sequence encoding a naturally virus infection-resistant receptor, wherein the method comprises:

obtaining one or more spermatogonial stem cells (SSCs) of a male animal that has an infection-susceptible endogenous receptor nucleic acid molecule;

providing a modification construct comprising an exogenous polycistronic inhibitory RNA nucleic acid sequence that suppresses the expression of the endogenous, virus infection-susceptible receptor, and further providing an exogenous nucleic acid sequence of the receptor from an alternative species having a different sequence than the endogenous virus infection-susceptible receptor; and

introducing the modification construct(s) into at least one of the SSCs, thereby obtaining at least one SSC comprising a nucleic acid molecule that suppresses the virus infection-susceptible endogenous receptor nucleic acid molecule and a second nucleic acid molecule that expresses a virus infection-resistant exogenous receptor nucleic acid molecule having a different sequence than the endogenous virus infection-susceptible receptor; and

introducing one or more modified SSCs into a reproductive organ of a male recipient animal; and optionally,

collecting the donor-derived, fertilization-competent, haploid male gametes produced by the male recipient.

In a preferred embodiment, the modification construct comprises a nucleic acid sequence encoding a polycistronic inhibitory RNA molecule, wherein the polycistronic inhibitory RNA molecule comprises multiple inhibitory RNA molecules, wherein the inhibitory RNA molecules suppress a virus infection-susceptible endogenous receptor nucleic acid sequence. In one embodiment, the modification construct also comprises an exogenous nucleic acid sequence comprising the intestinal receptor of an alternative species which is resistant to virus infection.

In one embodiment, the nucleic acid sequence encoding the polycistronic inhibitory RNA molecule and the nucleic acid sequence encoding the virus infection-resistant receptor of an alternative species are present on one construct.

In one embodiment, the nucleic acid sequence encoding the polycistronic inhibitory RNA molecule and the nucleic acid sequence encoding the virus infection-resistant receptor of an alternative species are present on different constructs.

In one embodiment, the genome of at least one modified SSC comprises a nucleic acid molecule comprising a nucleic acid sequence encoding a polycistronic inhibitory RNA and a nucleic acid sequence encoding a virus infection-resistant version of a receptor, which version originates from an alternative species, which species is not susceptible to infection by such virus.

In a further embodiment of the subject invention, extracellular domains of endogenous virus infection-susceptible receptors are expressed in the mammary gland of an animal and secreted into milk. In a preferred embodiment, nucleic acid sequences encoding the extracellular domain of an endogenous virus infection-susceptible receptor are linked to a signal sequence that enables polypeptide processing for secretion and are expressed under the control of a promoter that regulates expression in the mammary epithelium of a mammal.

In one embodiment the subject invention provides a method for improving animal resistance to infection by viruses, wherein the method comprises:

obtaining one or more spermatogonial stem cells (SSCs) of a male animal that has an infection-susceptible endogenous receptor nucleic acid molecule;

providing a modification construct comprising a nucleic acid sequence encoding the extracellular domain decoy of an infection-susceptible endogenous receptor linked to a signal sequence that enables polypeptide processing for secretion and is expressed under the control of a promoter that regulates expression in the mammary epithelium of a mammal; and

introducing the modification construct(s) into at least one of the SSCs, thereby obtaining at least one SSC comprising a nucleic acid molecule that expresses an extracellular domain decoy of an infection-susceptible endogenous receptor; and

introducing one or more modified SSCs into a reproductive organ of a male recipient animal; and optionally,

collecting the donor-derived, fertilization-competent, haploid male gametes produced by the male recipient; wherein female animals derived from the male recipient express a virus-binding decoy receptor in the milk providing protection for their off-spring.

In one embodiment, the nucleic acid sequence encoding the polycistronic inhibitory RNA molecule, the nucleic acid sequence encoding the virus infection-resistant receptor of an alternative species, and the nucleic acid encoding the extracellular domain decoy of the virus-infection-susceptible endogenous receptor are present on the modification construct.

In one embodiment, the nucleic acid sequence encoding the polycistronic inhibitory RNA molecule, the nucleic acid sequence encoding the virus infection-resistant receptor of an alternative species, and the nucleic acid encoding the extracellular domain decoy of the virus-infection-susceptible endogenous receptor are present on different constructs.

In one embodiment, outbred off-spring of homozygous females, which off-spring are heterozygous for the virus infection-resistant intestinal receptor in their intestinal epithelium will receive decoy versions of the virus infection-susceptible receptors through the milk and will be substantially protected against intestinal virus infection.

In a further embodiment of the subject invention, at least one nucleic acid sequence encoding an extracellular domain of an endogenous virus infection-susceptible receptor is introduced into a vector (e.g. a bacterium or virus), wherein the vector is then administered to an animal and the extracellular domains of the endogenous, virus infection-susceptible receptors expressed by the vector bind viruses in the intestinal lumen of the animal and prevent infection of the animal. Preferably the vector is administered orally.

In one embodiment of the subject invention, an expression construct is introduced into at least one bacterial cell, which expression construct contains at least one nucleic acid sequence encoding at least one extracellular domain of an endogenous virus infection-susceptible receptor and a signal sequence, in which at least one nucleic acid sequence is operably linked to a promoter, which promoter drives expression of the at least one extracellular domain of the endogenous virus infection-susceptible receptor, wherein the at least one extracellular domain of the endogenous virus infection-susceptible receptor, by virtue of the signal sequence, is secreted into the extracellular space surrounding the bacterial cell.

In another embodiment of the subject invention, a bacterial cell comprising an expression construct comprising a nucleic acid sequence encoding a signal sequence and an extracellular domain of a virus infection-susceptible receptor operably linked to a constitutive promoter, continuously expresses and secretes extracellular domains of the virus infection-susceptible receptors into the extracellular space.

In another embodiment of the subject invention, a bacterial cell comprises an expression construct comprising a nucleic acid sequence encoding a signal sequence and an extracellular domain of a virus infection-susceptible receptor operably linked to an inducible promoter, wherein the bacterial cell expresses and secretes extracellular domains of the virus infection-susceptible receptors in the presence of an inducing agent.

In a preferred embodiment of the subject invention, the expression construct comprises multiple nucleic acid sequences encoding multiple extracellular domains of multiple endogenous virus infection-susceptible receptors, each nucleic acid sequence also comprising a signal sequence, and each nucleic acid sequence operably linked to a constitutive or inducible promoter, wherein the expression of multiple extracellular domains of multiple endogenous virus infection-susceptible receptors occurs either continuously or in the presence of an inducing agent.

In preferred embodiments of the subject invention, the bacteria are of a species naturally occurring in the intestinal tract of animals, including the genus of Lactobacillus and the genus of Bifidobacterium.

### DEFINITIONS

As used herein, the term “expression construct” refers to a combination of nucleic acid sequences that provides for transcription of an operably linked nucleic acid sequence. Expression constructs of the invention also generally include regulatory elements that are functional in the intended host cell or virus in which the expression construct is to be expressed. Regulatory elements include promoters, transcription termination sequences, translation termination sequences, enhancers, and polyadenylation elements.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

As used herein, the term “operably linked” refers to a juxtaposition of the components described wherein the components are in a relationship that permits them to function in their intended manner. In general, operably linked components are in contiguous relation. Sequence(s) operably-linked to a coding sequence may be capable of effecting the replication, transcription and/or translation of the coding sequence. For example, a coding sequence is operably-linked to a promoter when the promoter is capable of directing transcription of that coding sequence.

A “coding sequence” or “coding region” is a polynucleotide sequence that is transcribed into mRNA and/or translated into a polypeptide. For example, a coding sequence may encode a polypeptide of interest. The boundaries of the coding sequence are determined by a translation start codon at the 5′-terminus and a translation stop codon at the 3′-terminus.

The term “promoter,” as used herein, refers to a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

In addition to a promoter, one or more enhancer sequences may be included such as, but not limited to, cytomegalovirus (CMV) early enhancer element and an SV40 enhancer element. Additional included sequences are an intron sequence such as the beta globin intron or a generic intron, a transcription termination sequence, and an mRNA polyadenylation (pA) sequence such as, but not limited to, SV40-pA, beta-globin-pA, the human growth hormone (hGH) pA and SCF-pA.

In one embodiment, the expression construct comprises polyadenylation sequences, such as polyadenylation sequences derived from bovine growth hormone (BGH) and SV40.

The term “polyA” or “p(A)” or “pA” refers to nucleic acid sequences that signal for transcription termination and mRNA polyadenylation. The polyA sequence is characterized by the hexanucleotide motif AAUAAA. Commonly used polyadenylation signals are the SV40 pA, the human growth hormone (hGH) pA, the beta-actin pA, and beta-globin pA. The sequences can range in length from 32 to 450 bp. Multiple pA signals may be used.

The terms “expression vector” and “transcription vector” are used interchangeably to refer to a vector that is suitable for use in a host cell (e.g., a subject's cell) and contains nucleic acid sequences that direct and/or control the expression of exogenous nucleic acid sequences.

Expression includes, but is not limited to, processes such as transcription, translation, and RNA splicing, if introns are present. Vectors useful according to the present invention include plasmids, viruses, BACs, YACs, and the like. Particular viral vectors illustratively include those derived from adenovirus, adeno-associated virus and lentivirus.

The term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

The term “recombinant” is used to indicate a nucleic acid construct in which two or more nucleic acids are linked and which are not found linked in nature.

The term “nucleic acid” as used herein refers to RNA or DNA molecules having more than one nucleotide in any form including single-stranded, double-stranded, oligonucleotide or polynucleotide.

The term “nucleotide sequence” is used to refer to the ordering of nucleotides in an oligonucleotide or polynucleotide in a single-stranded form of nucleic acid.

The term “expressed” refers to transcription of a nucleic acid sequence to produce a corresponding mRNA and/or translation of the mRNA to produce the corresponding protein.

Expression constructs can be generated recombinantly or synthetically or by DNA synthesis using well-known methodology.

The term “regulatory element” as used herein refers to a nucleotide sequence which controls some aspect of the expression of an operably linked nucleic acid sequence. Exemplary regulatory elements illustratively include an enhancer, an internal ribosome entry site (IRES), an intron, an origin of replication, a polyadenylation signal (pA), a promoter, a transcription termination sequence, and an upstream regulatory domain, which contribute to the replication, transcription, and post-transcriptional processing of a nucleic acid sequence. Those of ordinary skill in the art are capable of selecting and using these and other regulatory elements in an expression construct with no more than routine experimentation.

In one embodiment, the construct of the present invention comprises an internal ribosome entry site (IRES). In one embodiment, the expression construct comprises kozak consensus sequences.

A “gene” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a coding sequence for any polypeptide or fragment thereof, a functioning version of a malfunctioning endogenous molecule or a malfunctioning version of a normally-functioning endogenous molecule. An exogenous molecule can also be the same type of molecule as an endogenous molecule but be derived from a different species than the species the endogenous molecule is derived from. For example, a human nucleic acid sequence may be introduced into a cell line originating from a hamster or mouse.

An “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP DNA-binding domain and a cleavage domain) and fusion nucleic acids (for example, a nucleic acid encoding the fusion protein described supra).

“Complement” or “complementary sequence” means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′. This invention encompasses complementary sequences to any of the nucleotide sequences claimed in this invention.

### Construct Design and Delivery

In one embodiment, the expression construct further comprises an excisable selection marker. Examples of selection markers useful according to the present invention include, but are not limited to, antibiotic resistance, fluorescent cell sorting marker, magnetic cell sorting marker, and any combination thereof. Suitable selection marker genes are known in the art, including but not limited to, nucleic acid molecules encoding proteins that mediate antibiotic resistance (e.g., ampicillin resistance, neomycin resistance, G418 resistance, and puromycin resistance), nucleic acid molecules encoding colored or fluorescent or luminescent proteins (e.g., green fluorescent protein, enhanced green fluorescent protein, red fluorescent protein, and luciferase), and nucleic acid molecules encoding proteins that mediate enhanced cell growth and/or gene amplification (e.g., dihydrofolate reductase). Epitope tags include, for example, one or more copies of FLAG, His, myc, Tap, HA or any detectable amino acid sequence.

The selection marker can be excisable by any recombinase (e.g., Piggyback™, Cre-Loxp recombinase, and Flp recombinase). Vector designs of Piggyback™, Cre-Loxp recombinase, Flp recombinase for excision of nucleic acid sequences are known in the art.

If desired, the vector may optionally contain flanking nucleic sequences that direct site-specific homologous recombination. The use of flanking DNA sequences to permit homologous recombination into a desired genetic locus is known in the art. At present it is preferred that up to several kilobases or more of flanking DNA corresponding to the chromosomal insertion site be present in the vector on both sides of the encoding sequence (or any other sequence of this invention to be inserted into a chromosomal location by homologous recombination) to assure precise replacement of chromosomal sequences with the exogenous DNA. See e.g. Deng et al, 1993, Mol. Cell. Biol 13(4):2134-40; Deng et al, 1992, Mol Cell Biol 12(8):3365-71; and Thomas et al, 1992, Mol Cell Biol 12(7):2919-23. It should also be noted that the cell of this invention may contain multiple copies of the gene of interest.

In one embodiment, the expression construct is introduced into the SSCs using a site-specific nuclease. Site-specific nucleases useful according to the present invention include, but are not limited to, transcription activator-like effector nucleases (TALENs), zinc-finger nucleases (ZFNs), and/or clustered regulatory interspaced short palindromic repeat (CRISPR)/Cas-based RNA-guided DNA endonucleases. TAL-effector nucleases are a class of nucleases that allow sequence-specific DNA cleavage, making it possible to perform site-specific gene editing.

Site-specific genome-editing materials and methods are known in the art. In certain embodiments, a site-specific nuclease is introduced to the host cell that is capable of causing a double-strand break near or within a genomic target site, which greatly increases the frequency of homologous recombination at or near the cleavage site. In certain embodiments, the recognition sequence for the nuclease is present in the host cell genome only at the target site, thereby minimizing any off-target genomic binding and cleavage by the nuclease.

In one embodiment, the site-specific nuclease recognizes a target sequence. In one embodiment, the site-specific nuclease is engineered to cleave a pre-determined nucleic acid sequence from the endogenous nucleic acid molecule, wherein the pre-determined sequence is located near the endogenous dominantly acting nucleic acid sequence.

Site-specific nucleases can be introduced into the SSCs using any method known in the art. In one embodiment, the site-specific nuclease enzymes are introduced directly into SSCs. In another embodiment, the present invention involves administering a nucleic acid molecule encoding a site-specific nuclease into the SSCs. In one embodiment, the nucleic acid molecule encoding the SSCs is in an expression vector. In one embodiment, the expression vector comprises a nucleic acid molecule encoding a site-specific nuclease.

The site-specific nuclease can be introduced into the SSCs before, during (or simultaneously), and/or after the administration of the correction vector to the SSCs.

### Target Animals

The animals that can be made resistant to viral infection in accordance with the subject invention can be of any species, including, but not limited to, mammalian species including, but not limited to, domesticated and laboratory animals such as dogs, cats, mice, rats, guinea pigs, and hamsters; livestock such as horses, cattle, pigs, sheep, goats, ducks, geese, and chickens; primates such as apes, chimpanzees, orangutans, humans, and monkeys; fish; amphibians such as frogs and salamanders; reptiles such as snakes and lizards; and other animals such as fox, weasels, rabbits, mink, beavers, ermines, otters, sable, seals, coyotes, chinchillas, deer, muskrats, and possum.

In certain embodiments, the animals are from any family of Equidae, Bovidae, Canidae, Felidae, and Suidae. In one embodiment, the animal is not a human. In one specific embodiment, the animal is a pig.

### Nuclease-Mediated Site-Specific Genome Editing

Methods of site-specific genome editing are known in the art. In certain embodiments, the present invention uses transcription activator-like effector nucleases (TALENs), zinc-finger nucleases (ZFNs), and/or clustered regulatory interspaced short palindromic repeat (CRISPR)/Cas-based RNA-guided DNA endonucleases for site-specific genome editing, all of which are known in the art. See Gaj et al., ZFN, TALEN, and CRISPR/Cas-Based Methods for Genome Engineering, Trends in Biotechnology, July 2013, Vol. 31, No. 7, which is hereby incorporated by reference in its entireties.

TALENs (transcription activator-like effector nucleases) are fusions of the nuclease (such as FokI) cleavage domain and DNA-binding domains derived from TALE proteins. TALEs contain multiple 33-35-amino-acid repeat domains that each recognizes a single base pair. TALENs can induce double-strand breaks that activate DNA damage response pathways and enable custom alteration.

ZFNs (zinc-finger nucleases) are fusions of the nonspecific DNA cleavage domain from a restriction endonuclease (such as FokI) with zinc-finger proteins. ZFN dimers induce target DNA double-strand breaks that stimulate DNA damage response pathways. The binding specificity of the designed zinc-finger domain directs the ZFN to a specific genomic site. ZFNickases (zinc-finger nickases) are ZFNs that contain inactivating mutations in one of the two nuclease (such as FokI) cleavage domains. ZFNickases make only single-stranded DNA breaks and induce HDR without activating the mutagenic NHEJ pathway.

ZFNs are engineered double-strand break inducing agents comprised of a zinc finger DNA binding domain and a double strand break inducing agent domain. Engineered ZFNs consist of two zinc finger arrays (ZFAs), each of which is fused to a single subunit of a non-specific endonuclease, such as the nuclease domain from the FokI enzyme, which becomes active upon dimerization. Typically, a single ZFA consists of 3 or 4 zinc finger domains, each of which is designed to recognize a specific nucleotide triplet (GGC, GAT, etc.). In certain embodiments, ZFNs composed of two “3-finger” ZFAs are capable of recognizing an 18 base pair target site; an 18 base pair recognition sequence is generally unique, even within large genomes such as those of humans and plants. By directing the co-localization and dimerization of two FokI nuclease monomers, ZFNs generate a functional site-specific endonuclease that creates a double-stranded break (DSB) in DNA at the targeted locus.

Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496.

CRISPR/Cas (CRISPR associated) (clustered regulatory interspaced short palindromic repeats) systems are loci that contain multiple short direct repeats, and provide acquired immunity to bacteria and archaea. CRISPR systems reply on crRNA and tracrRNA for sequence-specific silencing of invading foreign DNA. Three types of CRISPR systems exist: in type II systems, Cas9 serves as an RNA-guided DNA endonuclease that cleaves DNA upon crRNA-tracrRNA target recognition.

crRNA: CRISPR RNA base pairs with tracrRNA to form a two-RNA structure that guides the Cas9 endonuclease to complementary DNA sites for cleavage.

A double-stranded break (DSB) is a form of DNA damage that occurs when both DNA strands are cleaved. DSBs can be products of TALENs, ZFNs, and CRISPR)/Cas9 action.

Homology-directed repair (HDR) is a template-dependent pathway for DSB repair. By supplying a homology-containing donor template along with a site-specific nuclease, HDR faithfully inserts the donor molecule at the targeted locus. This approach enables the insertion of single or multiple transgenes, as well as single nucleotide substitutions.

NHEJ (nonhomologous end joining) is a DSB repair pathway that ligates or joins two broken ends together. NHEJ does not use a homologous template for repair and thus typically leads to the introduction of small insertions and deletions at the site of the break.

PAMs (protospacer adjacent motifs) are short nucleotide motifs that occur on crRNA and are specifically recognized and required by Cas9 for DNA cleavage.

tracrRNA (transactivating chimeric RNA) is noncoding RNA that promotes crRNA processing and is required for activating RNA-guided cleavage by Cas9.

In one embodiment, the site-specific genome-editing method comprises contacting the host cell with one or more integration polynucleotides comprising an exogenous nucleic acid to be integrated into the genomic target site, and one or more nucleases capable of causing a double-strand break near or within the genomic target site. Cleavage near or within the genomic target site greatly increases the frequency of homologous recombination at or near the cleavage site.

In certain embodiments, a site-specific nuclease cleaves DNA in cellular chromatin, and facilitates targeted integration of an exogenous sequence (donor polynucleotide). In certain embodiments for targeted integration, one or more zinc finger or TALE DNA binding domains are engineered to bind a target site at or near the predetermined cleavage site, and a fusion protein comprising the engineered zinc finger or TALE DNA binding domain and a cleavage domain is expressed in a cell. Upon binding of the zinc finger or TALE DNA binding portion of the fusion protein to the target site, the DNA is cleaved, preferably via a double stranded break, near the target site by the cleavage domain. The presence of a double-stranded break facilitates integration of exogenous sequences as described herein via NHEJ mechanisms.

The exogenous (donor) sequence can be introduced into the cell prior to, concurrently with, or subsequent to, expression of the fusion protein(s).

“Recombination” refers to a process of exchange of genetic information between two polynucleotides. As used herein, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends:

A “cleavage domain” comprises one or more polypeptide sequences which catalyze activity for DNA cleavage.

A “cleavage half-domain” is a polypeptide sequence which, in conjunction with a second polypeptide (either identical or different), forms a complex having cleavage activity (preferably double-strand cleavage activity).

In one embodiment, the present invention employs markerless genomic integration of an exogenous nucleic acid using a site-specific nuclease. In one embodiment, an exogenous donor polynucleotide is introduced to a host cell, wherein the polynucleotide comprises a nucleic acid of interest (D) flanked by a first homology region (HR1) and a second homology region (HR2). HR1 and HR2 share homology with 5′ and 3′ regions, respectively, of a genomic target site (TS). A site-specific nuclease (N) is also introduced to the host cell, wherein the nuclease is capable of recognizing and cleaving a unique sequence within the target site. Upon induction of a double-stranded break within the target site by the site-specific nuclease, endogenous homologous recombination machinery integrates the nucleic acid of interest at the cleaved target site at a higher frequency as compared to a target site not comprising a double-stranded break.

Various methods are available to identify those cells having an altered genome at or near the target site without the use of a selectable marker. In some embodiments, such methods seek to detect any change in the target site, and include but are not limited to PCR methods, sequencing methods, nuclease digestion, e.g., restriction mapping, Southern blots, and any combination thereof.

Cleavage domains useful according to the present invention can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). Non limiting examples of homing endonucleases and meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

A recognition sequence is any polynucleotide sequence that is specifically recognized and/or bound by a double-strand break inducing agent. The length of the recognition site sequence can vary, and includes, for example, sequences that are at least 10, 12, 14, 16, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more nucleotides in length.

In some embodiments, the recognition sequence is palindromic, that is, the sequence on one strand reads the same in the opposite direction on the complementary strand. In some embodiments, the cleavage site is within the recognition sequence. In other embodiments, the cleavage site is outside of the recognition sequence. In some embodiments, cleavage produces blunt end termini. In other embodiments, cleavage produces single-stranded overhangs, i.e., “sticky ends,” which can be either 5′ overhangs, or 3′ overhangs.

In some embodiments of the methods provided herein, one or more of the nucleases is a site-specific recombinase. A site-specific recombinase, also referred to as a recombinase, is a polypeptide that catalyzes conservative site-specific recombination between its compatible recombination sites, and includes native polypeptides as well as derivatives, variants and/or fragments that retain activity, and native polynucleotides, derivatives, variants, and/or fragments that encode a recombinase that retains activity. The recognition sites range from about 30 nucleotide minimal sites to a few hundred nucleotides. Any recognition site for a recombinase can be used, including naturally occurring sites, and variants.

In some embodiments of the methods provided herein, one or more of the nucleases is a transposase. Transposases are polypeptides that mediate transposition of a transposon from one location in the genome to another. Transposases typically induce double strand breaks to excise the transposon, recognize subterminal repeats, and bring together the ends of the excised transposon. In some systems other proteins are also required to bring together the ends during transposition. Examples of transposons and transposases include, but are not limited to, the Ac/Ds, Dt/rdt, Mu-Ml/Mn, and Spm(En)/dSpm elements from maize, the Tam elements from snapdragon, the Mu transposon from bacteriophage, bacterial transposons (Tn) and insertion sequences (IS), Ty elements of yeast (retrotransposon), Tal elements from Arabidopsis (retrotransposon), the P element transposon from Drosophila (Gloor, et al., (1991) Science 253:1110-1117), the Copia, Mariner and Minos elements from Drosophila, the Hermes elements from the housefly, the PiggyBack™ elements from Trichplusia ni, Tc1 elements from C. elegans, and IAP elements from mice (retrotransposon).

The Cre-LoxP recombination system is a site-specific recombination technology useful for performing site-specific deletions, insertions, translocations, and inversions in the DNA of cells or transgenic animals. The Cre recombinase protein (encoded by the locus originally named as “causes recombination”) consists of four subunits and two domains: a larger carboxyl (C-terminal) domain and a smaller amino (N-terminal) domain. The loxP (locus of X-over P1) is a site on the Bacteriophage P1 and consists of 34 bp. The results of Cre-recombinase-mediated recombination depend on the location and orientation of the loxP sites, which can be located cis or trans. In case of cis-localization, the orientation of the loxP sites can be the same or opposite. In case of trans-localization, the DNA strands involved can be linear or circular. The results of Cre recombinase-mediated recombination can be excision (when the loxP sites are in the same orientation) or inversion (when the loxP sites are in the opposite orientation) of an intervening sequence in case of cis loxP sites, or insertion of one DNA into another or translocation between two molecules (chromosomes) in case of trans loxP sites. The Cre-LoxP recombination system is known in the art, see, for example, Andras Nagy, Cre recombinase: the universal reagent for genome tailoring, Genesis 26:99-109 (2000).

The Lox-Stop-Lox (LSL) cassette prevents expression of the transgene in the absence of Cre-mediated recombination. In the presence of Cre recombinase, the LoxP sites recombine, and the stop cassette is deleted. The Lox-Stop-Lox (LSL) cassette is known in the art. See, Allen Institute for Brain Science, Mouse Brain Connectivity Altas, Technical White Paper: Transgenic Characterization Overview (2012).

### Materials for Practicing the Methods of the Subject Invention

The present invention also provides materials for replacing a nucleic acid sequence encoding a virus infection-resistant receptor in animals. In one embodiment, the present invention provides a composition comprising an expression construct, a site-specific nuclease, and, optionally, one or more SSCs of a male animal whose genome contains a nucleic acid sequence encoding a virus infection-resistant receptor.

Optionally, the composition may also comprise any material useful for performing the expression method of the present invention. The kit may also comprise, e.g., vectors, culture media, preservatives, diluents, components necessary for detecting the detectable agent (e.g., a selectable marker).

### Bacterial Delivery of Decoy Receptors

In certain embodiments, bacterial cells are generated that express and secrete decoy receptors comprised of extracellular domains of endogenous virus infection-susceptible receptors using well-known molecular biology techniques. The bacteria can be of any genus non-pathogenic to the animal. Examples of preferred bacterial cells of the subject invention are Lactobacillus acetotolerans, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus arizonensis, Lactobacillus aviarius, Lactobacillus bifermentans, Lactobacillus brevis, Lactobacillus buchneri, Lactobacillus casei, Lactobacillus coelohominis, Lactobacillus collinoides, Lactobacillus coryniformis subsp. coryniformis, Lactobacillus coryniformis subsp. torquens, Lactobacillus crispatus, Lactobacillus curvatus, Lactobacillus cypricasei, Lactobacillus delbrueckii subsp. bulgaricus, Lactobacillus delbrueckii subsp delbrueckii, Lactobacillus delbrueckii subsp. lactis, Lactobacillus durianus, Lactobacillus equi, Lactobacillus farciminis, Lactobacillus ferintoshensis, Lactobacillus fermentum, Lactobacillus fomicalis, Lactobacillus fructivorans, Lactobacillus frumenti, Lactobacillus fuchuensis, Lactobacillus gallinarum, Lactobacillus gasseri, Lactobacillus graminis, Lactobacillus hamsteri, Lactobacillus helveticus, Lactobacillus helveticus subsp. jugurti, Lactobacillus heterohiochii, Lactobacillus hilgardii, Lactobacillus homohiochii, Lactobacillus intestinalis, Lactobacillus japonicus, Lactobacillus jensenii, Lactobacillus johnsonii, Lactobacillus kefiri, Lactobacillus kimchii, Lactobacillus kunkeei, Lactobacillus leichmannii, Lactobacillus letivazi, Lactobacillus lindneri, Lactobacillus malefermentans, Lactobacillus mali, Lactobacillus maltaromicus, Lactobacillus manihotivorans, Lactobacillus mindensis, Lactobacillus mucosae, Lactobacillus murinus, Lactobacillus nagelii, Lactobacillus oris, Lactobacillus panis, Lactobacillus pantheri, Lactobacillus parabuchneri, Lactobacillus paracasei subsp. paracasei, Lactobacillus paracasei subsp. pseudoplantarum, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus psittaci, Lactobacillus reuteri, Lactobacillus rhamnosus, Lactobacillus ruminis, Lactobacillus sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus thermophilus, Lactobacillus thermotolerans, Lactobacillus vaccinostercus, Lactobacillus vaginalis, Lactobacillus versmoldensis, Lactobacillus vitulinus, Lactobacillus vermiforme, Lactobacillus zeae and Bifidobacterium adolescentis, Bifidobacterium aerophilum, Bifidobacterium angulatum, Bifidobacterium animalis, Bifidobacterium asteroides, Bifidobacterium bifidum, Bifidobacterium boum, Bifidobacterium breve, Bifidobacterium catenulatum, Bifidobacterium choerinum, Bifidobacterium coryneforme, Bifidobacterium cuniculi, Bifidobacterium dentium, Bifidobacterium gallicum, Bifidobacterium gallinarum, Bifidobacterium indicum, Bifidobacterium longum, Bifidobacterium longum bv Longum, Bifidobacterium longum bv. Infantis, Bifidobacterium longum bv. Suis, Bifidobacterium magnum, Bifidobacterium merycicum, Bifidobacterium minimum, Bifidobacterium pseudocatenulatum, Bifidobacterium pseudolongum, Bifidobacterium pseudolongum subsp. globosum, Bifidobacterium pseudolongum subsp. pseudolongum, Bifidobacterium psychroaerophilum, Bifidobacterium pullorum, Bifidobacterium ruminantium, Bifidobacterium saeculare, Bifidobacterium scardovii, Bifidobacterium subtile, Bifidobacterium thermoacidophilum, Bifidobacterium thermoacidophilum subsp. suis, Bifidobacterium thermophilum, Bifidobacterium urinalis.

Expression constructs comprising nucleic acid sequences encoding one or multiple extracellular domains of one or multiple endogenous virus infection-susceptible receptors will be introduced into bacterial cells using one of several delivery methods known in the art.

Delivery via a virus can utilize, for example, any of the conventional viral based systems that are described below.

### Delivery Methods

The nucleic acids (including nucleic acid molecules encoding a site-specific nuclease or the expression construct) as described herein can be introduced into a cell or virus using any suitable method. Nucleases can also be introduced directly into the cells. For example, two polynucleotides, each comprising sequences encoding one of the aforementioned polypeptides, can be introduced into a cell, and when the polypeptides are expressed and each binds to its target sequence, cleavage occurs at or near the target sequence. Alternatively, a single polynucleotide comprising sequences encoding both fusion polypeptides, is introduced into a cell. Polynucleotides can be DNA, RNA or any modified forms or analogues of DNA and/or RNA.

In certain embodiments, one or more proteins can be cloned into a vector for transfection of cells. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties.

In certain embodiments, the nucleases and exogenous sequences are delivered in vivo or ex vivo in cells. Non-viral vector delivery systems for delivering polynucleotides to cells include DNA plasmids, naked nucleic acid, and nucleic acid complexed with a delivery vehicle such as a liposome or poloxamer.

Conventional viral based systems for the delivery of nucleases and nucleic acid molecules include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 by inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)).

Methods of non-viral delivery of nucleic acids in vivo or ex vivo include electroporation, lipofection, microinjection, biolistics, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipid:nucleic acid conjugates, naked DNA, artificial virions, viral vector systems (e.g., retroviral, lentivirus, adenoviral, adeno-associated, vaccinia and herpes simplex virus vectors as described in WO 2007/014275 for delivering proteins comprising ZFPs) and agent-enhanced uptake of DNA.

Lipofection is described in for example, U.S. Pat. No. 5,049,386; U.S. Pat. No. 4,946,787; and U.S. Pat. No. 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

Additional exemplary nucleic acid delivery systems include those provided by Amaxa Biosystems (Cologne, Germany), Maxcyte, Inc. (Rockville, Md.) and BTX Molecular Delivery Systems (Holliston, Mass.) and Copernicus Therapeutics Inc., (see for example U.S. Pat. No. 6,008,336).

Microinjection: Direct microinjection of DNA into various cells, including egg or embryo cells, has also been employed effectively for transforming many species. In the mouse, the existence of pluripotent embryonic stem (ES) cells that are culturable in vitro has been exploited to generate transformed mice. The ES cells can be transformed in culture, then micro-injected into mouse blastocysts, where they integrate into the developing embryo and ultimately generate germline chimeras. By interbreeding heterozygous siblings, homozygous animals carrying the desired gene can be obtained.

### Spermatogonial Stem Cell Transfer

Methods for performing spermatogonial stem cell transfer are known in the art.

In one embodiment, the SSC transfer method useful according to the present invention comprises:
providing spermatogonial stem cells (SSCs) from a male donor animal;
   introducing the donor SSCs into a reproductive organ of a sterile male recipient animal, whereby the sterile male recipient produces donor-derived, fertilization-competent, haploid male gametes; and optionally,
collecting the donor-derived, fertilization-competent, haploid male gametes produced by the sterile male recipient.

In certain embodiments, the SSC transfer method uses sterile, hybrid male recipient animals or sterile male recipient animals that have been genetically modified to have heritable male sterility.

In one embodiment, the recipient male animal is genetically modified such that it has an intact spermatogenic compartment but cannot perform spermatogenesis.

In certain embodiments, the sterile recipient animal can be produced via deletion or inactivating mutations of genes including, but not limited to, Deleted-in-Azoospermia like (DAZL); protamine genes (e.g., PRM1, PRM2) associated with DNA packaging in the sperm nucleus; genes in the azoospermia factor (AZF) region of the Y chromosome (such genes include, but are not limited to, USP9Y); and genes associated with male meiosis (such genes include, but are not limited to, HORMA domain-containing protein 1 (HORMAD1)). In another embodiment, the sterile recipient animal can be produced via genetic mutation(s) associated with sertoli cell-only syndrome (such genetic mutation includes mutations in USP9Y).

In one specific embodiment, the recipient male animal is genetically modified such that it does not express functional Deleted-in-Azoospermia like (DAZL) protein. In one specific embodiment, the recipient male animal is genetically modified such that the DAZL gene is deleted.

In one specific embodiment, the recipient male animal is genetically modified such that the DAZL gene does not encode functional DAZL protein.

As used herein, an inactivating mutation refers to any mutation (genetic alteration of a DNA molecule) that leads to an at least 30% reduction of function of the protein encoded by the DNA molecule. In one embodiment, the present invention provides a method for effecting spermatogonial stem cell (SSC) transfer, wherein the method comprises:
providing spermatogonial stem cells (SSCs) from a male donor animal;
introducing the donor SSCs into a reproductive organ of a sterile, hybrid male recipient animal, whereby the sterile, hybrid male recipient produces donor-derived, fertilization-competent, haploid male gametes; and optionally,
collecting the donor-derived, fertilization-competent, haploid male gametes produced by the sterile, hybrid male recipient.

The term “hybrid animal,” as used herein, refers to a crossbred animal with parentage of two different species. Hybrid male animals are usually sterile and cannot produce fertilization-competent, haploid male gametes. Examples of hybrid animals include, but are not limited to, mules (a cross between a horse and a donkey), ligers (a cross between a lion and a tiger), yattles (a cross between a yak and a buffalo), dzo (a cross between a yak and a bull), and hybrid animals that are crosses between servals and ocelots/domestic cats.

In another embodiment, the SSC transfer method useful according to the present invention comprises:
providing spermatogonial stem cells (SSCs) from a male donor animal;
introducing the donor SSCs into a reproductive organ of a genetically-modified, sterile male recipient animal, whereby the sterile male recipient produces donor-derived, fertilization-competent, haploid male gametes, and wherein the sterile male recipient animal is genetically modified such that it has an intact spermatogenic compartment but cannot perform spermatogenesis; and optionally,
collecting the donor-derived, fertilization-competent, haploid male gametes produced by the sterile male recipient.

In another embodiment, the present invention provides a method for effecting spermatogonial stem cell (SSC) transfer, wherein the method comprises:
providing spermatogonial stem cells (SSCs) from a male donor animal;
introducing the donor SSCs into a reproductive organ of a genetically-modified male recipient animal whereby the recipient produces donor-derived, fertilization-competent, haploid male gametes, wherein the recipient animal is genetically modified such that the native male gametes produced by the recipient animal express at least one detectable biomarker label; optionally,
distinguishing the native male gametes produced by the recipient animal from the donor-derived male gametes produced by the recipient animal based on the detectable biomarker label; and optionally,
collecting donor-derived, fertilization-competent, haploid male gametes produced by the recipient animal.

In one specific embodiment, the native male gametes produced by the recipient animal express at least one detectable cell surface biomarker (such as cell-surface antigen tag(s)).

In one embodiment, native male gametes produced by the recipient animal express luminescent proteins. In one embodiment, native male gametes produced by the recipient animal are distinguished from the donor-derived male gametes produced by the recipient animal by flow sorting, such as fluorescence activated cell sorting (FACS) and magnetic-activated cell sorting (MACS).

In one embodiment, the genetically-modified recipient male animal comprises a reporter gene for expression on the cell surface of native male gametes. In certain embodiments, the reporter gene encodes a luminescent protein.

The term “luminescent protein,” as used herein, refers to a protein that emits light. Luminescent proteins useful according to the present invention include, but are not limited to, fluorescent proteins including, but not limited to, green fluorescent protein, yellow fluorescent protein, cyan fluorescent protein, and red fluorescent protein; and phosphorescent proteins. Fluorescent proteins are members of a class of proteins that share the unique property of being self-sufficient to form a visible wavelength chromophore from a sequence of three amino acids within their own polypeptide sequence. A variety of luminescent proteins, including fluorescent proteins, are publicly known. Fluorescent proteins useful according to the present invention include, but are not limited to, the fluorescent proteins disclosed in U.S. Pat. No. 7,160,698, U.S. Application Publication Nos. 2009/0221799, 2009/0092960, 2007/0204355, 2007/0122851, 2006/0183133, 2005/0048609, 2012/0238726, 2012/0034643, 2011/0269945, 2011/0223636, 2011/0152502, 2011/0126305, 2011/0099646, 2010/0286370, 2010/0233726, 2010/0184116, 2010/0087006, 2010/0035287, 2007/0021598, 2005/0244921, 2005/0221338, 2004/0146972, and 2001/0003650, all of which are hereby incorporated by reference in their entireties.

In one embodiment, donor SSCs are introduced into the testis of the male recipient animal.

In one embodiment, male gametes produced by the recipient animal are sperm.

In one embodiment, the donor spermatogonial stem cells (SSCs) embody a genetic background of interest. In one specific embodiment, the donor animal is from the Genus of Sus, including but not limited to, Sus scrofa domesticus (domestic pig).

In certain embodiments, the recipient animal can be adult animals or immature animals. In one embodiment, the recipient animal is in puberty.

In a further embodiment, the present invention further comprises the step of fertilizing an egg from an animal species of interest with the donor-derived, fertilization-competent, haploid male gamete produced by the recipient animal. Methods of fertilization of eggs are known in the art, and include, but are not limited to, intracytoplasmic sperm injection (ICSI) and round spermatid injection (ROSI).

Parentages of the recipient hybrid animal, the recipient animal, and/or the donor animal can be of any animal species including, but not limited to, species of pigs; horses; cattle; sheep; cats; mice; rats; wolves; coyotes; dogs; chinchillas; deer; muskrats; lions; tigers; hamsters; goats; ducks; geese; chickens; primates such as apes, chimpanzees, orangutans, monkeys; and humans.

In certain embodiments, one or both parentages of the recipient hybrid animal, the recipient animal, and/or the donor animal can be of any vertebrates, including fish, amphibians, birds, and mammals. In certain embodiments, one or both parentages of the recipient hybrid animal, the recipient animal, and/or the donor animal are not a human.

In certain embodiments, one or both parentages of the recipient hybrid animal, the recipient animal, and/or the donor animal can be from any family of Suidae, Equidae, Bovidae, Canidae, and Felidae.

Mammalian spermatogonial stem cells (SSCs) self-renew and produce daughter cells that commit to differentiate into spermatozoa throughout adult life of the male. SSCs can be identified by functional assays known in the art, such as transplantation techniques in which donor testis cells are injected into the seminiferous tubules of a sterile recipient.

In one embodiment, donor spermatogonial stem cells can be cryopreserved and/or cultured in vitro. Frozen spermatogonial stem cells can be grown in vitro and cryopreserved again during the preservation period.

SSCs can be cultured in serum-containing or serum-free medium. In one embodiment, the cell culture medium comprises Dulbecco's Modified Eagle Medium (DMEM), and optionally, fetal calf serum.

In certain embodiments, SSC culture medium can comprise one or more ingredients including, but not limited to, glial cell-derived neurotrophic factor (GDNF), fibroblast growth factor-2 (FGF2), leukemia inhibitory factor (LIF), insulin-like growth factor-I (IGF-I), epidermal growth factor (EGF), stem cell factor (SCF), B27-minus vitamin A, Ham's F12 nutrient mixture, 2-mercaptoethanol, and L-glutamine.

Methods for transplanting spermatogonial stem cells into recipient reproductive organs (such as, the testis) are known in the art. Transplantation can be performed by direct injection into seminiferous tubules through microinjection or by injection into efferent ducts through microinjection, thereby allowing SSCs to reach the rete testis of the recipient. The transplanted spermatogonial stem cells adhere to the tube wall of the recipient seminiferous tubules, and then differentiate and develop into spermatocytes, spermatids and spermatozoa, and finally mature following transfer to the epididymis.

Methods for the introduction of one or more SSCs into a recipient male also include injection into the vas deferens and epididymis or manipulations on fetal or juvenile testes, techniques to sever the seminiferous tubules inside the testicular covering, with minimal trauma, which allow injected cells to enter the cut ends of the tubules. Alternatively, neonatal testis (or testes), which are still undergoing development, can be used.

### EXAMPLES

Following are examples that illustrate procedures and embodiments for practicing the invention. These examples should not be construed as limiting.

### Example 1

The target receptor in livestock can be replaced with the receptor from another species with either no known intestinal viruses, or with which the livestock is unlikely to come in contact.

For instance, the target receptor for PED is a gene called ANPEP. Although knocking out the ANPEP receptor would make pigs immune, this is not a preferred embodiment because the ANPEP receptor is important for protein absorption in the gut, and knockouts would have deficient protein absorption. Instead, the pig ANPEP gene can be replaced with ANPEP from Bactrian camels, woolly mammoths, tree sloths, giant pandas, or any other species that, because of rarity or environment does not have known intestinal viruses.

Pigs with two copies of the replacement ANPEP will be immune to PED, because the disease only recognizes the porcine version of the ANPEP receptor, which will be missing in these animals. Pigs with one copy might have partial protection.

If the viral target sequence is specifically known, solely the viral target sequence can be modified or replaced with that from another species.

### Example 2

Livestock can be modified such that a decoy version of the target receptor is expressed in milk. As occurs naturally in humans for some noroviruses, this protects the offspring from virus infection by binding up virus before it has a chance to infect the intestinal lining.

The decoy version of the target receptor has several modifications:
(1) The decoy lacks the transmembrane domain needed for stable integration into the cell surface.
(2) The decoy contains a signal sequence that enables processing for secretion.
(3) The decoy is modified to have reduced binding to its natural target—for instance, protein, in the case of ANPEP—without removing the target sequences for the virus.
(4) If the viral target sequence is known specifically, the decoy can lack sections of the receptor not necessary for viral binding.
(5) The decoy is driven by a promoter specific to cells that secrete proteins into milk (several of these have been published over the past decades).

This approach has the advantage that even heterozygous females will provide substantial protection for their offspring.

### Example 3

Decoy receptors, as described above, can be produced through any of several methods of protein synthesis, including production in the milk as above, production in yeast, production in bacteria or production through artificial synthesis methods. The decoy receptors are purified and stored, and stored decoy receptors can be consumed during an outbreak, to reduce susceptibility. Alternatively, bacteria comprising nucleic acid sequences encoding signal sequences and decoy receptors can be orally administered to animals, wherein the decoy receptors expressed and secreted by the ingested bacteria bind viruses present in the intestinal lumen and prevent virus binding to endogenous virus infection susceptible receptors and infection of the animal. This method allows protection for non-genetically modified animals, including humans.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures, tables, and sequences, to the extent they are not inconsistent with the explicit teachings of this specification.

We claim:

1. A nucleic acid molecule comprising:
an exogenous, inhibitory polycistronic RNA coding sequence, operably linked to a promoter, wherein the exogenous inhibitory polycistronic RNA coding sequence encodes multiple inhibitory RNA molecules that interfere with the expression of an endogenous virus infection-susceptible receptor and thereby reduce and/or prevent expression of the endogenous virus infection-susceptible receptor; and/or
an exogenous nucleic acid molecule, operably linked to a promoter, wherein the exogenous nucleic acid molecule encodes a receptor derived from another species than the animal and wherein the receptor of the other species is resistant to virus infection; and/or
an exogenous nucleic acid molecule that encodes the extracellular domain of an endogenous virus infection-susceptible receptor, wherein expression of the exogenous, extracellular domain-encoding nucleic acid sequence leads to secretion of decoy receptors.

2. A cell of a non-human transgenic animal comprising:
a nucleic acid molecule encoding a virus infection-susceptible receptor; and
a nucleic acid molecule of claim 1.

3. The nucleic acid molecule of claim 1, wherein the exogenous nucleic acid molecule is operably linked to a promoter that induces mammary gland expression, and is operably linked to a signal sequence that induces secretion of the exogenous nucleic acid molecule such that expression of the exogenous nucleic acid sequence leads to secretion in the milk of the animal.

4. A method for improving animal resistance to infection by viruses, wherein the method comprises:
obtaining one or more spermatogonial stem cells (SSCs) of a male animal that has an endogenous nucleic acid encoding a virus infection-susceptible receptor;
providing a modification construct comprising a nucleic acid molecule of claim 1;
introducing the modification construct(s) into at least one of the SSCs, thereby obtaining at least one SSC comprising said exogenous nucleic acid molecule; and
introducing one or more of said SSCs into a reproductive organ of a male recipient animal; and, optionally;
collecting the donor-derived, fertilization-competent, haploid male gametes produced by the male recipient.

5. The method, according to claim 4, wherein the exogenous inhibitory polycistronic RNA and the exogenous nucleic acid sequence encoding for a virus infection-resistant receptor from another species are delivered via a single construct.

6. A bacterial cell comprising a nucleic acid molecule of claim 1.

7. A method for improving animal resistance to infection by a virus, wherein the method comprises introducing one or more bacterial cells of claim 6 into the digestive tract of an animal.

8. A method for improving animal resistance to infection by viruses, wherein said method comprises interfering with virus uptake by (1) reducing virus binding to a receptor on a cell of the animal, (2) introducing a decoy receptor gene into the genome of the animal to induce secretion of a decoy receptor, (3) administering one or more decoy receptors to the animal, and/or (4) administering a vector to the animal wherein the vector produces a decoy receptor.

9. A modified animal resistant to infection by a virus, wherein the animal
a) expresses a mutant viral receptor to which the virus cannot bind,
b) expresses a homolog of a viral receptor from a species of animal that the virus cannot infect,
c) expresses a decoy receptor, and/or
d) comprises a vector that produces a decoy receptor.

10. The animal of claim 9, wherein the animal is a genetically engineered animal.

11. The genetically engineered animal of claim 10, wherein the mutant viral receptor comprises one or more mutations in the amino acid residues that constitute the binding site for the virus and the virus cannot bind to the mutant receptor.

12. The genetically engineered animal of claim 10, wherein the genetically engineered animal expresses a decoy receptor through one or more copies of the genes encoding the decoy receptor in to the genome of the animal.

13. The genetically engineered animal of claim 10, wherein the genetically engineered animal expresses a decoy receptor specifically in the cells of the mucus membrane.

14. The genetically engineered animal of claim 10, wherein the genetically engineered animal is a pig.

15. A method of producing an animal of claim 9, the method comprising modifying the animal so that the animal:
a) expresses a mutant viral receptor to which the virus cannot bind,
b) expresses a homolog of a viral receptor from a species of animal which the virus cannot infect,
c) expresses a decoy receptor, and/or
d) comprises a vector that produces a decoy receptor.

16. The method of claim 15, comprising genetically modifying the animal.

17. The method of claim 15, comprising introducing one or more copies of the genes encoding the mutant viral receptor, or a homolog of a viral receptor, into the genome of the animal.

18. The method of claim 15, comprising introducing one or more copies of the genes encoding a decoy receptor into the genome of the animal.

19. The method of claim 15, comprising introducing into the genome of the animal one or more genes encoding the decoy receptor that are under the control of a promoter specific for the cells of the mucus membrane.


SEQUENCE LISTING (1 sequence)

SEQ ID NO: 1 - protein, 967 aa, Camelus bactrianus
MGKGFYISKALGILGILLGVAAVATIIALSVVYAQEKNKNAECATVAPSTTPSTTPSTSSATTLDQSKPWNQYRLPTTLLPDSYNVKLRPYLTPNDKGLYIFKGTSTVRFICKEPTNVIIIHSKKLNYTTTQEHMVVLRGVGGTQAPSIDRTELVELTQYLVVHLKGSLEAGRMYEMDTEFEGELADDLAGFYRSEYMEGNVKKVLATTQMQAPDARKSFPCFDEPAMKATFNITLIHPKDLTALSNMLPKGPSVPLEGDPNWNVTEFETTPKMSTYLLAFIVSEFTCVEGKSANDVLIRIWARPNATAEGHGIYALNKTGPILDFFAHHYATSYPLPKSDQIALPDFNAGAMENWGLVTYRENALLFDPLSSSISNKERVVTVIAHELAHQWFGNLVTLAWWNDLWLNEGFASYVEYLGADYAEPNWNLKDLIVTNDVYPVMAVDALASSHPLTTPAKEVNTPAQISEMFDTISYSKGASVIRMLSDFLTEELFKKGLASYLNAFAYQNTTYLDLWEHLQKAVNNQTSIQLPDTVRAIMDRWTLQMGFPVITVDTKTGDISQKHFLLDPDSNVTRPSEFNYLWIVPISSIRNGTPQAHYWLQGQEKAQDNLFKTGTNEWVLLNVNVTGYYQVNYDDNNWRMIQSQLQIDPSAIPVINRAQVIYDGFNLANAHIVSVTLALNNTLFLNKETEYMPWQAALNSLSYFKLMFDRSEVYGPMKKYLRKQVEPLFQHFESITKNWTERPQNLMDQYNEINAISTACSNGLSKCEELAKTLFSSWMKDPENNPIHPNLRSTIYCNAIAQGGQEEWDFAWEQFQKAQLVNEADKLRSGLACSNEVWLLNRYLSYTLNPDLIRKQDATSTINSIASNVIGQPLAWDFVQSNWKKLFQDYGGGSFSFSNLIQAVTRRFSTEQELQELEQFKQNNMDVGFGSGTRALEQALEKTKANIKWVKENKEVVLQWFTENS